(19) United States
(12) Patent Application Publication
(10) Pub. No.: US 2018/0147084 A1
(43) Pub. Date: May 31, 2018

(54) Device and System for Improved Breathing

(71) Applicant: Airway Technologies, LLC, Carrollton, TX (US)

(72) Inventors: W. Keith Thornton, Dallas, TX (US); Alastair E. McAuley, Warkworth (NZ)

(21) Appl. No.: 15/580,225

(22) PCT Filed: Jun. 7, 2016
(86) PCT No.: PCT/US16/36198

§ 371 Date: Dec. 6, 2017

Related U.S. Application Data
(60) Provisional application No. 62/172,340, filed on Jun. 8, 2015.

(51) Int. Cl. A61F 5/56; A61M 16/06; A61M 16/04; A61M 16/00

(52) U.S. Cl. CPC A61F 5/566; A61M 16/0605; A61M 16/049; A61M 2230/205; A61M 2230/06; A61M 2205/52; A61M 2205/3375; A61M 2016/0042; A61M 2016/0027; A61M 2230/42; A61M 2230/435; A61M 2210/0637

(57) ABSTRACT

In some embodiments, a system for improving a user's breathing includes a mask configured to be positioned on a user's face to deliver gas to the user, a first oral appliance arch configured to be worn in the user's mouth on the user's mandibular dentition, a first tension element coupled to the first oral appliance arch, an adjustment device coupled to the mask, and a control system. The adjustment device is configured to receive the first tension element and to adjust the position of the first tension element to adjust the forward position of the first oral appliance arch, such that the when the mask is positioned on the user's face and the first oral appliance arch is worn on a user's mandibular dentition, the forward position of the user's mandible is adjusted.

### TECHNICAL FIELD OF THE INVENTION

This invention relates generally to medical and dental devices, and more particularly to devices and systems for improving a user's breathing.

### BACKGROUND

Many people experience difficulty sleeping because of breathing problems. These problems may result in snoring or the more serious condition of sleep apnea. One treatment for sleep breathing disorders involves the use of dental devices for extending forward the lower jaw of the patient. These devices operate to more fully open the breathing passageway, thereby allowing for easier breathing, whether that breathing be through the nose or through the mouth. Another treatment for sleep breathing disorders involves the use of masks to deliver air to users. These masks may also be used to deliver oxygen or other gases to a user.

### SUMMARY OF THE DISCLOSURE

In accordance with the teachings of the present disclosure, devices and systems are provided which may reduce or eliminate disadvantages and problems associated with prior art devices.

In certain embodiments, a system for improving a user's breathing includes a mask configured to be positioned on a user's face to deliver gas to the user, a first oral appliance arch configured to be worn in the user's mouth on the user's mandibular dentition, a first tension element coupled to the first oral appliance arch, an adjustment device coupled to the mask, and a control system. The adjustment device is configured to receive the first tension element and to adjust the position of the first tension element to adjust the forward position of the first oral appliance arch, such that the when the mask is positioned on the user's face and when the first oral appliance arch is worn on the user's mandibular dentition, the forward position of the user's mandible is adjusted. The control system may be configured to control the adjustment device to adjust the position of the first tension element. The mask may be a multi-chamber mask comprising a nasal chamber and an oral chamber, the nasal chamber separated from the oral chamber by a partition. The system may include a second oral appliance arch configured to be worn in the user's mouth on the user's maxillary dentition and a second tension element coupled to the second oral appliance arch, wherein the adjustment device is further configured to receive the second tension element to position or hold the mask on the user's face. The control system may be configured to control the adjustment device in response to receiving signals representative of measurement data for one or more of the following: flow volume for exhaled gases of the user; pressure of exhaled gases of the user; breathing rate of the user; oxygen levels of gases exhaled by the user; blood oxygen levels of the user; pulse rate of the user; vibrations of the mask; vibrations of the adjustment device; and sound.

In certain embodiments, a system for improving a user's breathing includes a mask configured to be positioned on a user's face to deliver gas to the user, an adjustment device coupled to the mask and configured to receive a first tension element and to adjust the position of the first tension element to adjust the forward position of a first oral appliance arch, such that the when the mask is positioned on the user's face and when the first oral appliance arch is worn on the user's mandibular dentition, the forward position of the user's mandible is adjusted. The adjustment device may include a motor and is configured to couple to a control system to control the adjustment of the first tension element. The mask may be a multi-chamber mask comprising a nasal chamber and an oral chamber, the nasal chamber separated from the oral chamber by a partition. The adjustment device may be configured to receive a second tension element coupled to a second oral appliance arch to position or hold the mask on the user's face. The adjustment device may include a release element configured to release at least the first tension element from the adjustment device. The adjustment device may include a manual adjuster configured to adjust the position of a tension element received by the adjustment device in response to manual actuation.

In certain embodiments, an adjustment device for use with a mask and an oral appliance arch includes a housing with a first opening to receive a first tension element coupled to a first oral appliance arch, an adjustment mechanism configured to engage the first tension element, and a motor configured to interact with the adjustment mechanism to adjust the position of the first tension element to adjust the forward position of the first oral appliance arch, wherein the adjustment mechanism and the motor are within the housing. The adjustment mechanism include (1) a gear configured to interact with notches in the first tension element, such that rotation of the gear will adjust the position of the first tension element; (2) a threaded element configured to interact with threads in the first tension element, such that rotation of the threaded element gear will adjust the position of the first tension element; and/or (3) a spool configured to receive a portion of the first tension element, such that rotation of the spool will adjust the position of the first tension element. The motor may be configured to couple to a control system to control the adjustment of the first tension element. The housing may have a second opening configured to receive a second tension element coupled to a second oral appliance arch. The adjustment device may include a release element configured to release at least the first tension element. The adjustment device may include a manual adjuster configured to adjust the position of a tension element received by the adjustment device in response to manual actuation.

In certain embodiments, a control system for use with a system to improve a user's breathing includes a receiver configured to receive signals representative of measurement data, a memory for storing program logic, and a processor configured to process the received signals and control an adjustment device in accordance with stored program logic to adjust the position of a tension element received within the adjustment mechanism.

Certain embodiments may provide one or more technical advantages. For example, certain embodiments may provide for adjustments to a user's mandibular position while a person is sleeping. As another example, certain embodiments may provide for automated adjustments to a user's mandibular position in response to one or more of a variety of measurements. As yet another example, certain embodiments may provide for an adjustable connection between a mask and an oral appliance that may be manually and/or automatically adjusted. Still other embodiments may provide a way for an off-the-shelf mask to be adjustably coupled to one or more oral appliance arches. Certain embodiments may provide some, none, or all of these advantages. Certain embodiments may provide one or more other technical advantages, one or more of which may be readily apparent to those skilled in the art from the figures, description, and claims included herein.

### BRIEF DESCRIPTION OF THE DRAWINGS

FIG. 1 illustrates an example system for improving a user's breathing.

FIG. 2A illustrates an example oral appliance arch.

FIG. 2B illustrates another view of an example oral appliance arch.

FIG. 2C illustrates another view of an example oral appliance arch.

FIG. 3A illustrates an example arched frame.

FIG. 3B illustrates another view of an example arched frame.

FIG. 4A illustrates an example moldable tray.

FIG. 4B illustrates another view of an example moldable tray.

FIG. 4C illustrates another view of an example moldable tray.

FIG. 5 illustrates an example custom-molded oral appliance arch.

FIG. 6 illustrates another view of an example system for improving a user's breathing.

FIG. 7A illustrates example components of an adjustment device

FIG. 7B illustrates example components of an adjustment device

FIG. 7C illustrates example components of an adjustment device

FIG. 8A illustrates an example adjustment device

FIG. 8B illustrates an example adjustment device

FIG. 8C illustrates an example adjustment device

FIG. 9A illustrates an example adjustment device

FIG. 9B illustrates an example adjustment device

FIG. 10 illustrates an example system for improving a user's breathing.

### DETAILED DESCRIPTION OF THE INVENTION

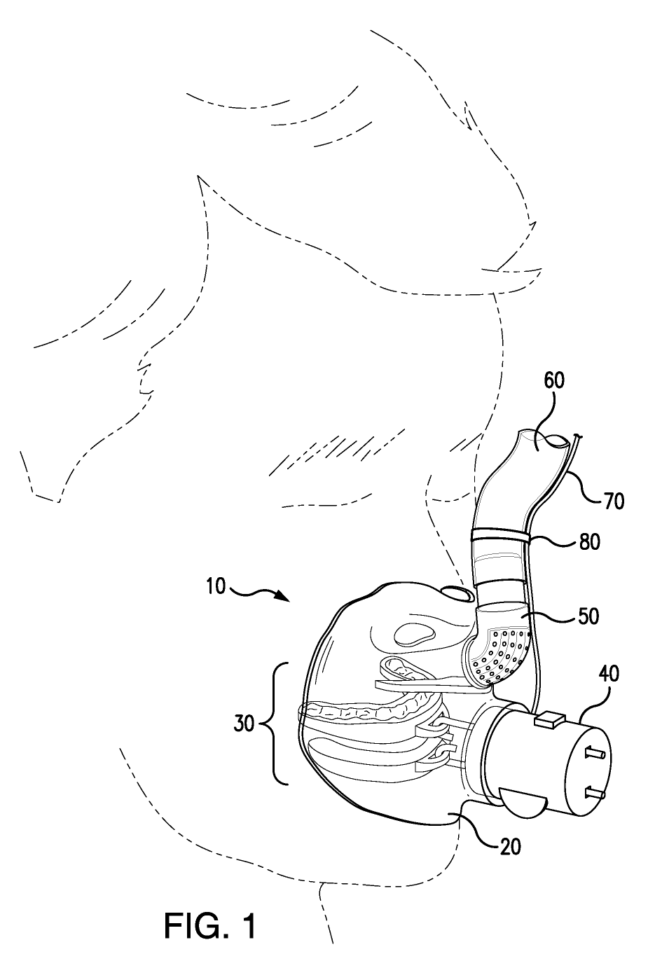

FIG. 1 illustrates an example system 10 for improving a user's breathing. In certain embodiments, system 10 includes mask 20, oral appliance 30, and adjustment device 40. In the embodiment shown, mask 20 is a multi-chamber mask coupled through an example fitting 50 to an example gas supply source 60 and adjustment device 40 is connected to cable 70 that is held next to a component of gas supply source 60 by support 80. In operation, system 10 may be used to administer air, oxygen, anesthetic, and/or another gas to a user. Fitting 50 may include any suitable structure to connect mask 20 to gas supply source 60. For example, fitting 50 may be an acrylic, male-type hose connector that couples to an opening into mask 20. As another example, fitting 50 may be a gasket surrounding an opening into mask 20. Gas supply source 60 may be, for example, a continuous positive air pressure (CPAP) system that supplies air or another gas at a positive pressure to help open the user's breathing passage and thereby improve the user's breathing. Such gasses may be supplied using alternating positive and ambient pressure, or any appropriate pattern of pressure to facilitate delivery of gas to the user.

In certain embodiments, mask 20 may be a multi-chamber mask. Although in alternative embodiments, mask 20 may be a single chamber mask, may be a combination mask that covers the mouth and includes nasal pillows, may be a nasal mask that covers portions of the nose, or may include nasal inserts or nasal pillows that may be positioned in and/or adjacent to the nostrils. Certain embodiments of the invention are not limited to the type of mask used with one or more other components of system 10. In certain embodiments, mask 20 may be custom formed to fit the user's unique facial structure and features. For example, mask 20 may be custom formed to fit at least the portion of the user's face surrounding the user's mouth and nostrils. Example custom formed masks may provide reduced leakage, increased comfort, and better performance. However, in certain embodiments system 10 may include masks that are not custom fitted. Example multi-chamber masks and methods of forming multi-chamber masks are disclosed in U.S. Pat. No. 7,909,035, incorporated herein by reference. Other example masks that may be included in system 10 and example methods of forming certain masks are disclosed in U.S. Pat. Nos. 6,857,428; 7,243,650; 8,871,251; 8,236,216; and in U.S. Patent Publication No. 2014/0053852, all of which are herein incorporated by reference.

In the embodiment shown, oral appliance 30 includes an upper arch and lower arch. Although in certain embodiments, oral appliance 30 may include a single upper or lower arch. In a particular embodiment, an arch of oral appliance 30 may include an arched frame that has been overmolded by a deformable material. Examples of oral appliances and methods of forming oral appliances are disclosed in U.S. Pat. Nos. 5,427,117; 7,174,895; 7,748.386; and 8,662,084; and in U.S. Patent Application Nos. 2014/0053852 and 2014/0290668, all of which patents and patent applications are incorporated herein by reference.

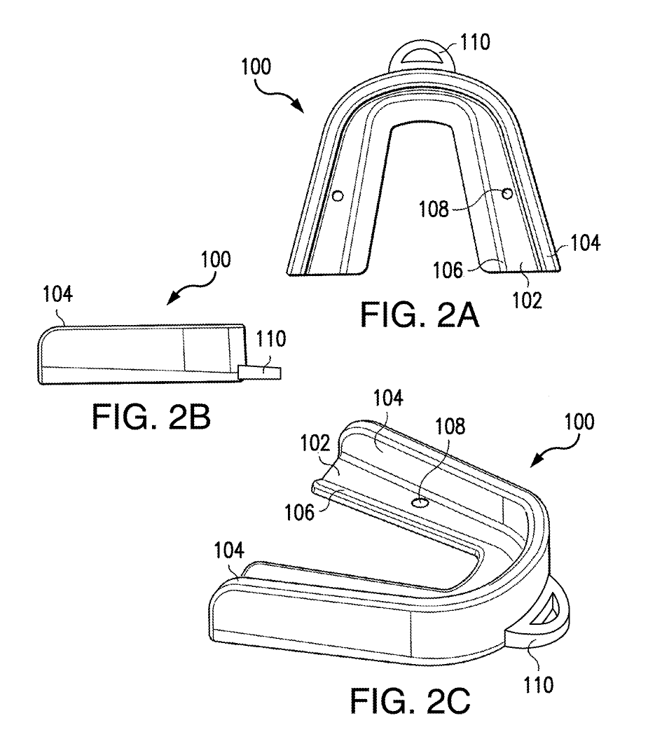

FIG. 2A illustrates an example oral appliance arch 100. FIGS. 2B and 2C illustrate alternate views of an example oral appliance arch 100. As shown in FIG. 2A, oral appliance arch 100 may include an occlusal surface 102, an outer rim 104, an inner rim 106, and recesses 108. Occlusal surface 102 may be configured to be placed proximate to the occlusal surface of a user's dental arch. Outer rim 104 may be configured to be positioned proximate to the labial surface of a user's dental arch. In certain embodiments, inner rim 106 may be configured to be positioned proximate to the lingual surface of a user's dental arch. In certain embodiments, inner rim 106 may lie mostly flat relative to the plane of occlusal surface 102 or may angle upward slightly. Such embodiments may make oral appliance arch 100 easier to slide into the user's mouth. In certain embodiments, inner rim 106 may be capable of being pushed upward or downward to engage with the lingual surface of the user's dental arch during the molding process.

As shown in FIG. 2A, in certain embodiments outer rim 104 may have a thickness greater than that of inner rim 106. For example, in certain embodiments, outer rim 104 may have a thickness of approximately 3 millimeters, while inner rim 106 may have a thickness of approximately 2 millimeters, although these dimensions are not required. Reduced thickness of inner rim 106 may allow oral appliance arch 100 to take up less space in the inner mouth area behind the teeth, which may allow the user to breath, swallow, and speak more easily and experience greater comfort. Reduced thickness of inner rim 106 may also help obviate any need to offer multiple sizes of oral appliance arch 100. In certain embodiments, reduced thickness of inner rim 106 may allow other medical and/or dental devices to be more easily inserted into the user's mouth. In certain embodiments, as shown in FIG. 2C, inner rim 106 may be shorter than outer rim 104. A shorter inner rim 106 may allow for easier insertion of oral appliance arch 100 into the user's mouth. A shorter inner rim 106 may also reduce the amount of moldable material in the inner mouth area, which may provide additional advantages as described above. In certain embodiments, distal portions of oral appliance arch 100 may have a reduced height, which may improve the fit of oral appliance arch 100 in the user's mouth.

As shown in FIG. 2A, in certain embodiments occlusal surface 102 may have one or more recesses 108, which may result from or facilitate clamping or otherwise holding in place arched frame 100 during manufacturing. One or more recesses 108 may also be used as index points to locate various components of oral appliance arch 100 during manufacturing.

As shown in FIG. 2A, in certain embodiments, oral appliance arch may include anterior structure 110. It should be appreciated that in certain embodiments anterior structure 110 may be fixed to oral appliance arch 100 or it may be removeably coupled to oral appliance arch 100. In certain embodiments, anterior structure 110 may provide a coupling point for a tension element that may couple, for example, to a mask or adjustment mechanism. In alternative embodiments, anterior structure 110 may include such tension element.

In certain embodiments, oral appliance arch 100 may be formed of acrylic, may formed using a multi-laminate process, and/or may be formed of a deformable thermoplastic material. In certain embodiments, oral appliance arch 100 may be an encased moldable tray. An example method of forming an encased moldable tray is described in U.S. Patent Application No. 2014/0290668, incorporated herein by reference. In certain embodiments, oral appliance arch 100 may be formed with an arched frame overmolded with a deformable material.

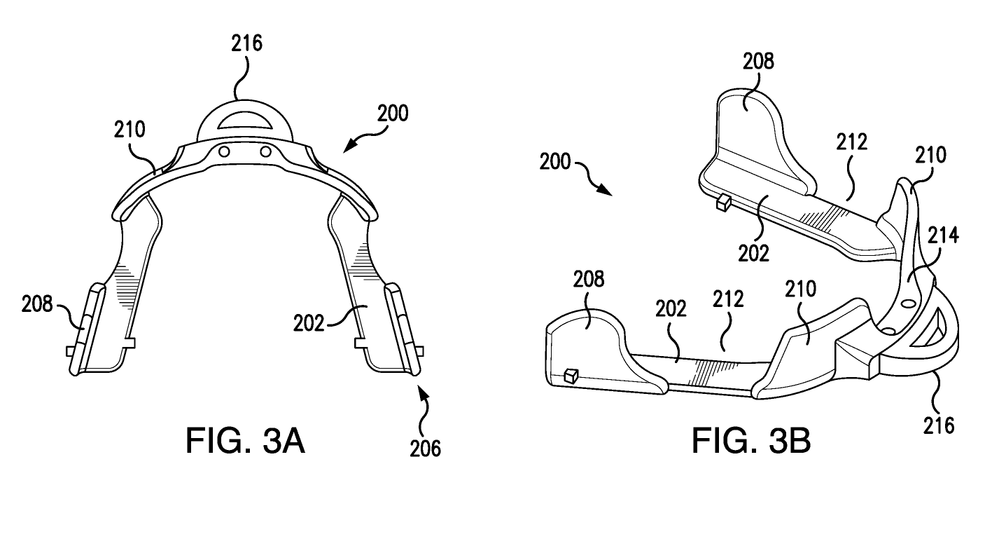

FIG. 3A illustrates an example arched frame 200. Arched frame 200 may include an occlusal surface 202 and a flange 206. In particular embodiments arched frame 200 may be configured to be positioned proximate to a user's dental arch, with occlusal surface 202 positioned proximate to the occlusal surface of the user's dental arch. In certain embodiments, occlusal surface 202 may be contiguous throughout the length of arched frame 200. In alternative embodiments, as shown in FIG. 3A, occlusal surface 202 may not be contiguous throughout the length of arched frame 200. For example, occlusal surface 202 may have a first portion configured to be positioned proximate to the user's left bicuspid and first molar; and have a second portion configured to be positioned proximate to the user's right bicuspid and first molar. As shown in FIG. 3A, in certain embodiments, occlusal surface 202 may not extend to the area proximate to the user's incisors. Certain embodiments in which the occlusal surface 202 is not contiguous throughout the length of arched frame 200 may allow for improved flexibility of arched frame 200. In certain embodiments, arched frame 200 may be capable of flexing inward and/or outward, allowing arched frame 200 to conform to a wider variety of dental arch shapes and sizes. Such embodiments may also improve the ability of arched frame 200 to accommodate the overlap of the user's maxillary and mandibular incisors, allowing the user's jaw to close more fully. In certain embodiments, occlusal surface 102 may have a thickness of approximately 1.5 millimeters, although other thicknesses may be used. As shown in FIG. 3A, in certain embodiments occlusal surface 202 may include an occlusal surface recess 204. When arched frame 200 is used with deformable material, occlusal surface recess 204 may allow the deformable material to form a closer mold of the user's teeth.

Flange 206 may run along the labial edge of arched frame 200. In certain embodiments, flange 206 may be contiguous throughout the length of arched frame 200. In alternative embodiments, as shown in FIG. 3A, flange 206 may not be contiguous throughout the length of the arched frame. For example, flange 106 may include a distal flange portion 208 and a mesial flange portion 210, separated by a flange recess 212. In certain embodiments, because flange 206 is positioned proximate to the labial surface of the user's dental arch, it may be pushed outward by the labial surface of the user's dental arch when arched frame 200 is inserted into the user's mouth during the molding process, allowing arched frame 200 to automatically flex and align with the user's dental arch, which may improve the ability of arched frame 200 to accommodate different dental arch sizes and shapes. In certain embodiments, flange recesses 212 may allow for improved flexibility of arched frame 200. In addition, when arched frame 200 is used with deformable material, flange recesses 212 may allow the deformable material to form an improved mold of the user's teeth. In some embodiments, flange recess 212 may improve the user's ability to press moldable material against their teeth during the molding process, which allow for improved dental molds. In certain embodiments, mesial flange portion 210 may allow for an improved mold when arched frame 200 is pressed toward the user's teeth during fitting. As shown in FIG. 3A, in certain embodiments mesial flange portion 210 may have a thickness greater than that of distal flange portion 208. For example, mesial flange portion 210 may have a thickness of approximately 3 millimeters and distal flange portion 208 may have a thickness of approximately 1.5 millimeters, although other thicknesses may be used. In such embodiments, the greater thickness of mesial flange portion 210 may improve the stability of arched frame 200 during flexion and may provide a more secure anchor point for other attached structures, such as anterior structure 216 shown in FIG. 3A.

As shown in FIG. 3A, certain embodiments may include anterior structure 216 which extends forward from mesial flange portion 210 in an anterior direction. In certain embodiments, anterior structure 216 may be fixed to arched frame 100, while in other embodiments anterior structure 216 may removeably coupled to arched frame 200. In certain embodiments, anterior structure 216 may provide a coupling point for a tension element that may couple, for example, to a mask or adjustment mechanism. In alternative embodiments, anterior structure 216 may include such tension element.

FIG. 3B illustrates an isometric view of an example arched frame 200. As shown in FIG. 3B, arched frame 200 may include anterior structure 216, occlusal surface 202 that is not contiguous throughout the length of arched frame 200, and flange 206 that includes a distal flange portion 208, a flange recess 212, a mesial flange portion 210, and a mesial flange recess 214. As shown in FIG. 3B, flange 206 may include mesial flange recess 214 located approximately at the midline of arched frame 200. In such embodiments, mesial flange recess 214 may allow for improved conformity with the shape of the user's mouth. In certain embodiments, the distal ends of arched frame 200 may extend approximately to the user's first molar when the frame is inserted into the user's mouth. In alternative embodiments, arched frame 200 may extend to the user's second molar or to the user's third molar.

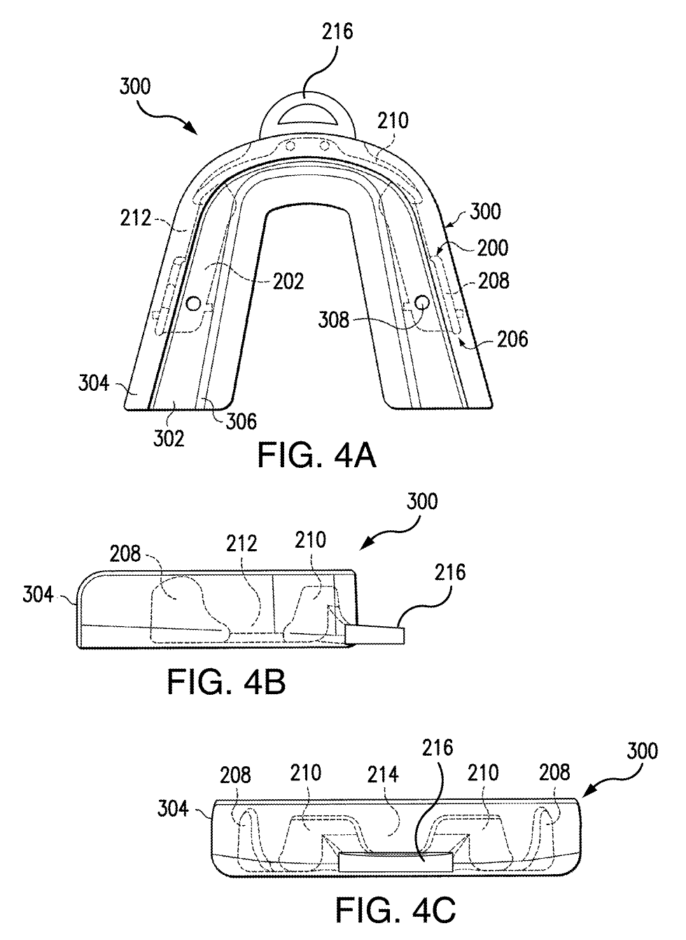

FIG. 4A illustrates an example moldable tray 300 substantially surrounding an example arched frame 200. As shown in FIG. 4A, example moldable tray 300 may include occlusal surface 302, outer rim 304, and inner rim 306; and arched frame 200 may include occlusal surface 202 and flange 206. As shown in FIG. 4A, in certain embodiments the labial edge of outer rim 304 may extend outward beyond the labial edge of flange 206. The lingual edge of inner rim 306 may also extend inward beyond the lingual edge of occlusal surface 202. As seen in FIG. 4A, in certain embodiments the distal end of moldable tray 300 may extend distally beyond the distal end of arched frame 200. In certain embodiments, the distal end of arched frame 200 may extend approximately to the user's first molar, while the distal end of moldable tray 300 extends approximately to the user's second or third molar. In alternative embodiments, the distal end of arched frame 200 may extend approximately to the user's second molar, while the distal end of moldable tray 300 extends approximately to the user's third molar. In still other embodiments, the distal ends of arched frame 200 and moldable tray 300 may be approximately coextensive.

In certain embodiments, when moldable tray 300 is oriented for placement on, for example, a user's maxillary arch, the superior surface of outer rim 304 may extend beyond the superior surface of flange 206 by approximately 2.5 millimeters while the inferior surface of moldable tray 300 may extend below the inferior surface of arched frame 200 by approximately 1.5 millimeters, although these dimensions are not required. In certain embodiments, moldable tray 300 may extend outward beyond the labial edge of arched frame 200 by approximately 1.5 millimeters, though other dimensions are possible. In certain embodiments, moldable tray 300 may extend inward beyond the lingual edge of arched frame 200 by approximately 1.5 millimeters, though other dimensions are possible.

In certain embodiments, flange 206 may help maintain the shape of outer rim 304. Moldable trays that substantially surround an arched frame may allow for reduced bulk between a user's incisors when the tray(s) are inserted into the user's mouth. By providing moldable trays with less material between the user's incisors, certain embodiments may allow users to close their mouths further, which may improve comfort and/or effectiveness. Furthermore, moldable trays that substantially surround arched frame may allow for mouth pieces where only the moldable material touches the inner surfaces of the user's mouth, such as the user's gums, lips, and tongue. Such moldable trays may also allow for improved molding to the user's front teeth. Having arched frame 200 substantially surrounded by moldable tray 300 may also reduce the chances of damage to arched frame 200 and may help hold any broken pieces of arched frame 200 in place, preventing any such broken pieces from contacting the user's mouth or entering the user's airway.

In certain embodiments, arched frame 200 may include apertures in occlusal surface 202 and/or flange 206, though such apertures are not required. Such apertures may allow the moldable material to flow through arched frame 200 during the molding process, which may provide greater stiffness following the molding process and may allow for improved alignment of arched frame 200 with moldable tray 300.

As shown in FIG. 4A, in certain embodiments occlusal surface 302 may have one or more recesses 308, which may result from clamping or otherwise holding in place arched frame 200 during an overmolding process. In certain embodiments, arched frame 200 may have a corresponding recess, which may allow for improved clamping and alignment during the manufacturing process.

In certain embodiments, moldable tray 300 may be composed of a material that can be heated to a temperature at which the material softens and becomes capable of being molded to a different shape. In certain embodiments, the material can be heated in hot water. In some embodiments, the temperature range at which the material softens may be approximately 40-80 degrees Celsius, although materials with other softening ranges may be used. In a particular embodiment, the target softening temperature may be approximately 60 degrees Celsius. In certain embodiments, this material may be a thermoplastic. Such thermoplastic materials may be heated to a temperature at which the thermoplastic becomes soft and moldable, at which point it may be molded to the shape of at least a portion of a user's dental arch and become at least temporarily fixed in that shape. As one example, moldable tray 300 may comprise a polycaprolactone polymer or other aliphatic polyester. In particular embodiments, the thermoplastic material may comprise a cross-linked polycaprolactone reinforced with an aramid fiber such as the short length aramid fiber sold by Dupont under the brand name Kevlar®. In certain embodiments, using polycaprolactone combined with Kevlar® may allow moldable tray 300 to soften at low temperatures and set hard at temperatures of approximately 60 degrees Celsius. In certain embodiments, using polycaprolactone combined with Kevlar® may improve the hardness of moldable tray 300 following the molding process, which may improve the ability of moldable tray 300 to hold its shape when being used to adjust the user's jaw position and/or hold a mask or other breathing device in place. In certain embodiments, this increased hardness may also improve the ability of moldable tray 300 to hold its shape for longer periods of time. For example, in certain embodiments, this may allow moldable tray 300 to substantially hold its shape for periods longer than approximately 1 month, though such period is not required. Using polycaprolactone combined with Kevlar® may also allow for thinner embodiments of moldable tray 300, which may allow moldable tray 300 to take up less space in the user's mouth. Examples of polycaprolactone combined with an aramid fiber, including Kevlar® and a variety of other fibers, are described in abandoned U.S. application Ser. No. 11/368,991, publication number U.S. 2007/0004993 A1, which is incorporated herein by reference. Such embodiments may provide an improved moldable material that better maintains its form when heated, providing increased viscosity which may prevent the material from flowing excessively around the user's teeth and/or getting stuck on the user's teeth during the molding process. Such embodiments may also possess increased strength after molding. In certain embodiments, the thermoplastic material may be cross-linked by radiation, which may create cross-linking of certain molecules to improve the material's shape retention characteristics and/or make the material better able to return to its original shape after reheating. In certain embodiments, radiation may be applied after arched frame 200 has been overmolded by moldable tray 300, but before being custom molded to the user, though this is not required. Cross-linking by radiation is further described in U.S. Pat. No. 5,415,623, which is incorporated herein by reference. In certain embodiments, the material may exhibit slight shrinkage after being molded to the user's dental arch. In particular embodiments, such shrinkage may be less than 1%. Slight shrinkage of the material following the molding process may allow for improved fit with the user's dental arch. In some embodiments, slight shrinkage of the material following the molding process may allow moldable tray 300 to have a “snap” fit with the user's dental arch.

In some embodiments, arched frame 200 may be primarily composed of a substantially rigid material, such as Nylon or any other material providing substantial rigidity while allowing moderate flexion. In certain embodiments, arched frame 200 may be composed of a material whose form does not substantially changed when heated to the temperature required to soften the moldable material of moldable tray 300. For example, in some embodiments, arched frame 200 may be composed of a material that substantially maintains its shape when heated up to at least 100 degrees Celsius. Such materials may include polycarbonate, Nylon, acrylonitrile butadiene styrene (ABS), or polyethylene. In certain embodiments, arched frame 200 may be composed of a semi-flexible material, for example liquid silicone rubber (LSR), approximately having a Shore 30-90 hardness, although this particular hardness is not required.

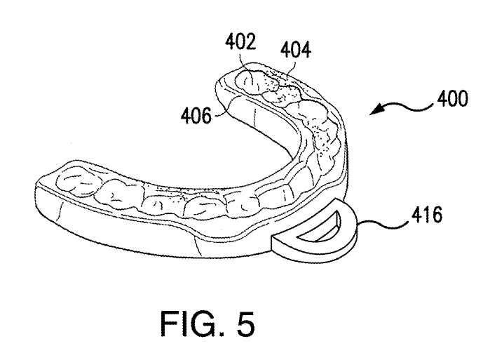

FIG. 5 illustrates an example oral appliance arch 400 that has been custom molded to fit a user's dentition. As shown in FIG. 5, oral appliance arch 400 includes occlusal surface 402, outer rim 404, inner rim 406, and anterior structure 416.

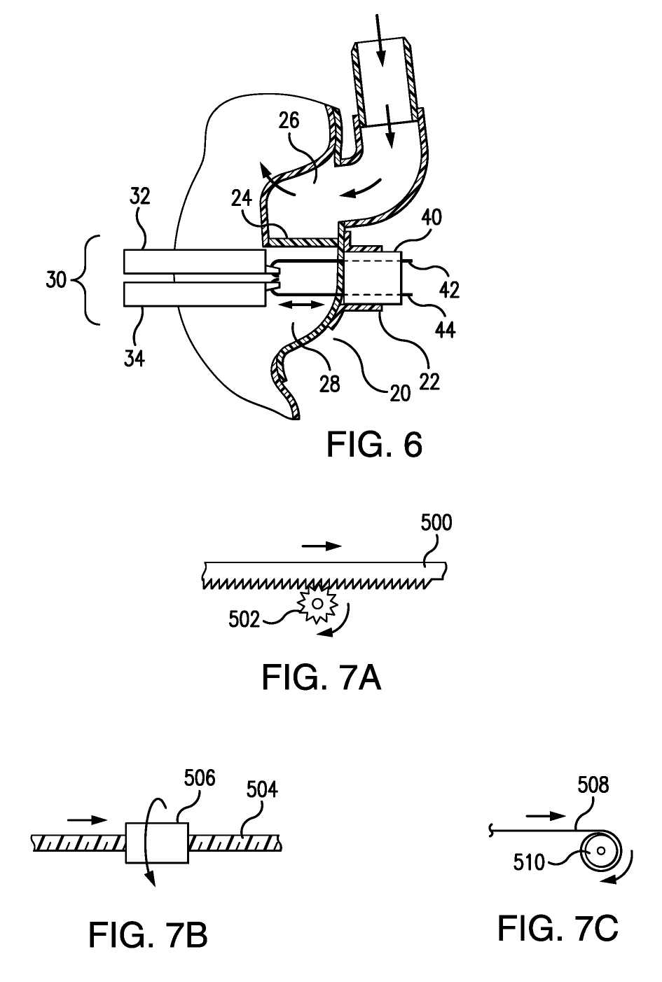

FIG. 6 illustrates another view of an example system 10 for improving a user's breathing. As discussed above in relation to FIG. 1, example system 10 includes mask 20, oral appliance 30, and adjustment device 40. In the embodiment shown, oral appliance 30 includes upper arch 32 and lower arch 34. Upper arch 32 is coupled to adjustment device 40 by tension element 42 and lower arch 44 is coupled to adjustment device 40 by tension element 44. Adjustment device 40 is coupled to mask 20 by coupler 22. In the embodiment shown, mask 20 is a multi-chamber mask that includes partition 24 separating a nasal chamber 26 from an oral chamber 28.

In operation, mask 20 may be positioned on the user's face and held in place, at least in part, due to tension being applied to tension element 42. The user may insert upper arch 32 and then tension may be applied to tension element 42 to position and hold mask 20 on the user's face. In certain embodiments, the tension applied to tension element 42 may be adjusted using adjustment device 40. In certain embodiments, the use of tension element 42 to position and hold mask 20 on the user's face may reduce or eliminate the need for straps to hold mask 20 on the user's face.

In operation, adjustment device 40 may be used to adjust the position of a user's mandible by adjusting the forward location of tension element 44. By moving the user's mandible forward it may open the user's breathing passages to allow for improved breathing. In operation, the user may insert lower arch 34 and then tension may be applied to tension element 44. As discussed further below, adjustment device may be calibrated and may adjust tension element 44 to improve the user's breathing.

FIGS. 7A through 7C illustrate example components of adjustment device 40. In certain embodiments, tension elements may be rigid, semi-rigid, or flexible and tension elements may have different shapes, as appropriate to provide sufficient tension and to interact with the components of tension device 40. For example, as illustrated in FIG. 7A, example tension element 500 may include a series of notches that may interact with a gear, such as gear 502. In this embodiment, the rotation of gear 502 will result in a translation of tension element 502. Once the proper position of tension element 500 is obtained, gear 502 may be locked or set, such that the position or tension does not change unintentionally. As another example, as illustrated in FIG. 7B, tension element 504 may be threaded and the threads of tension element 504 may interact with the internal threads (not shown) of element 506, such that rotation of element 506 will result in a translation of tension element 504. Once the proper position of tension element 504 is obtained, element 506 may be locked or set, such that the position or tension does not change unintentionally. As another example, as illustrated in FIG. 7C, tension element 508 may be a flexible element, such as a cable made of nylon or other material, and may be wound around spool 510 to adjust the length of tension element 508. In this example, rotation of spool 510 can adjust the length of tension element 508. In certain embodiments, spool 510 may be coupled to a spring element (not shown) to retract tension element and remove any excess length. In certain embodiment, a combination of these components may be used to adjust the length of multiple tension elements. In alternative embodiments, other known components may be used to adjust the position or length of one or more tension elements.

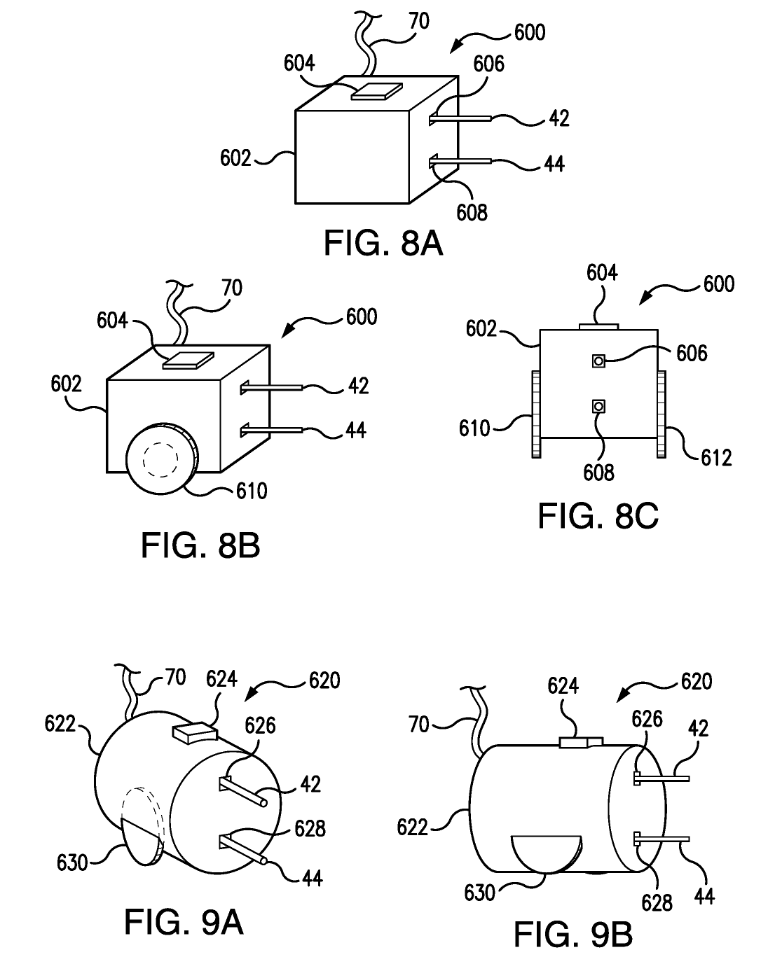

FIGS. 8A through 8C illustrates an example adjustment devices 600. In the embodiment shown in FIG. 8A, adjustment device 600 is coupled to cable 70 and includes housing 602, release element 604, and openings 606 and 608. In this embodiment, adjustment device interacts with tension elements 42 and 44. Tension element 42 may extend through opening 606 to allow for adjustment to tension element 42. Similarly, tension element 44 may extend through opening 608 to allow for adjustment to tension element 44.

Release element 604 may be used to release tension from one or more of tension elements 42 and 44. In certain embodiments, release element 604 may allow one or more of tension elements 42 and 44 to move freely within adjustment device 600. In certain embodiments, release element 604 may only affect a single tension element. In certain embodiments, adjustment device may have a separate release element 604 for each tension element. In a particular embodiment, release element 604 is a button. In operation, for example, depressing release element 604 may separate gear 502 from tension element 500. As another example, depressing release element 604 may release spool 510 to turn freely or to turn with only a spring load applied. Although, release element 604 has been described as a button, in alternative embodiments, release element may take other forms, such as a switch, touch sensor, dial, etc.

In the embodiment shown in FIG. 8B, adjustment device 600 includes a manual adjuster 610 that may be used to adjust the position or tension for a tension element, such as tension elements 42 and 44. In the embodiment shown, manual adjuster 610 is illustrated as a dial that can be rotated to adjust a tension element. In certain embodiments, as shown for example in FIG. 8C, adjustment device 600 may include a first manual adjuster 610 and a second manual adjuster 612. In this embodiment, separate manual adjusters may be used to separately adjust the position or tension for different tension elements. For example, manual adjustor 610 may be used to adjust tension element 42 and manual adjustor 612 may be used to adjust tension element 44, or vice versa. Alternatively, both adjusters 610 and 612 could be used to adjust a single tension element, thereby allowing a user to use either their left or right hand to make adjustments to that tension element. Although manual adjusters 610 and 612 are illustrated as dials, in alternative embodiments, one or more of manual adjustors may take other forms, such as slide actuators, touch sensors, levers, etc.

In certain embodiments, various components of adjustment device may be operated through the use of one or more motors within adjustment device 600. For example, in various embodiments one or more motors could be used to rotate gear 502, element 506, or spool 510 to adjust the position of one or more tension elements. In these embodiments, power for the one or more motors may be supplied through cable 70. Alternatively, power for the one or more motors may be supplied by a battery. In certain embodiments, the power to the one or more motors may be controlled in response to wireless signals received by a receiver included with adjustment device 600 and processed by a processor included within adjustment device 600. In addition or alternatively, power to the one or more motors may be controlled by a controller external to adjustment device 600 that controls the power provided to the one or more motors through cable 70. In certain embodiments, the use of one or more motors may be used as a substitute for, or in addition to, the use of one or more manual adjusters 610 and 612.

For example, the one or more motors may be controlled by a control system that includes a receiver, a memory, and a processor. The receiver may receive signals representative of measurement data (e.g., flow volume for exhaled gases of the user, pressure of exhaled gases of the user, breathing rate of the user, oxygen levels of gases exhaled by the user, blood oxygen levels of the user, pulse rate of the user, vibrations of the mask, vibrations of the adjustment device, and/or sound). The receiver may receive these signals wirelessly or through a wired connection. The memory may store program logic that is executed by the processor to control the one or more motors and/or the adjustment device. For example, the program logic may cause the processor to analyze the received signals and/or the measurement data to determine whether the position of the user's jaw should be adjusted. The processor may then cause the motor to operate, which may adjust the position of the tension element. Adjusting the tension element may adjust the position of the user's jaw.

In certain embodiments, the power to one or more motors of adjustment device 600 may be controlled in response to the receipt of information reflecting one or more of the following measurements: flow volume or pressure for the exhaled gases of the user, breathing rate of the user, the oxygen levels of the gases exhaled by the user, the blood oxygen levels of the user, the pulse rate of the user, and vibrations of the mask or adjustment device. As another example, the control of power to one or more motors of adjustment device 600 may be controlled in response to sound information. For example, in response to the detection of snoring by the user. In this example, upon detecting that the user is snoring, the motor could be controlled to move tension element 44 to adjust the forward position of the user's mandible to open the users breathing passages. These adjustments could be continued gradually until the snoring stops. The control of power to the one or more motors could be set to adjust the one or more tension elements according to a program. Alternatively, or additionally, the control of power to the one or more motors could be set to adjust the one or more tension elements in response to external input from, for example, a doctor, nurse, or other clinician.

In certain embodiments, the travel distance for one or more tension elements could be calibrated and set to ensure the safety and comfort of the user. Alternatively, or additionally, physical safety devices could be used to limit the movement of one or more of the tension elements. For example, a physical stop could be attached to tension element 44 to prevent tension element from moving forward beyond a certain distance.

In certain embodiments, adjustment device may include a position indicator, that may determine the orientation of the adjustment device relative to gravitational forces. In these embodiments, the orientation of adjustment device 600 may be used as an input to control the power to one or more motors of adjustment device 600 or for other purposes. In these embodiments, the orientation of adjustment device 600 may serve as a proxy for the orientation of the user as laying on their side, laying on their back, sitting up, etc. In these embodiments, as one example, the tension on tension element 44 may be released in response to an indication that the user is sitting up.

In the embodiments shown in FIGS. 8A through 8C, housing 602 has been drawn with a generally cubic form for simplicity. In alternative embodiments, the shape of an adjustment device housing could take many different forms to accommodate the various components and features described herein. As just one example, as shown in FIGS. 9A and 9B, an adjustment device housing could be generally cylindrical in form. In particular, FIGS. 9A and 9B illustrate an example adjustment device 620 coupled to cable 70 and including housing 622, release element 624, openings 626 and 628, and manual adjuster 630. In this embodiment, adjustment device 620 interacts with tension elements 42 and 44.

In addition to the components of example adjustment device 600, adjustment device 600 will include internal components to adjust one or more tension elements. Such additional elements may include one or more of the components illustrated in FIGS. 7A through 7C. In addition, in embodiments in which adjustment device 600 is automated, adjustment device 600 may include one or more motors to drive the adjustment components. In addition, adjustment device 600 may include additional shafts, gears, wheels, latches, and/or stops, known in the art to implement a manual or motor driven adjustment using components of the type illustrated in FIGS. 7A thorough 7B. Alternatively, rather than or in addition to using mechanical components to adjust tension elements, adjustment mechanism 600 may include pneumatic, hydraulic, electric, or magnetic actuators, or a combination thereof, to adjust one or more tension elements. Accordingly, those portions of the disclosure above that describe the use of one or more motors, would apply similarly to situations that utilize pneumatic, hydraulic, electric of magnetic actuators rather than using mechanical components and one or more motors.

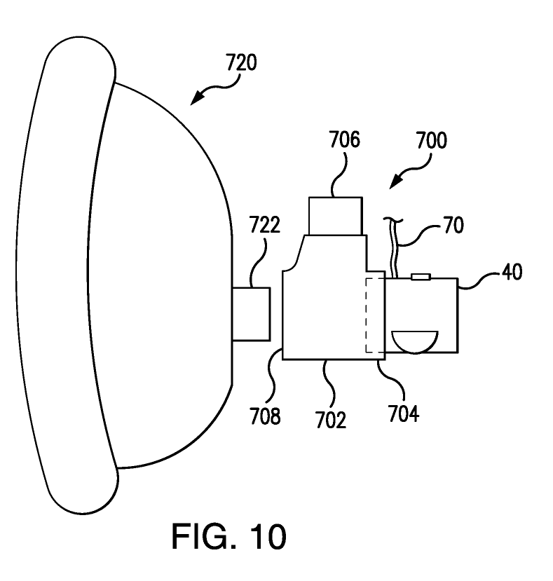

FIG. 10 illustrates an example system for improving a user's breathing. As illustrated in FIG. 10, system includes adjustment device 40, adapter 700, and mask 720. Adapter 700 includes couplers 704, 706, and 708. Coupler 704 is configured to receive at least a portion of adjustment device 40. Coupler 706 is configured to couple to a gas delivery system. For example, coupler 706 may be a male type connector, configured to be received by the end of a hose of a gas delivery system. Coupler 708 is configured to couple to a corresponding coupler 722 of mask 720. For example mask 720 may have a male type connector designed to connect mask 720 to a hose of a gas delivery system. In this embodiment, coupler 708 may be configured to receive the male type connector to couple adapter 700 to mask 720. In operation, adapter 700 may be used to couple adjustment device 40 to mask 720, without the need for mask 720 to be customized to couple to adjustment device 40. Thus, adapter 40 may be used with “off-the-shelf” masks and allow such masks to be connected to one or more oral appliance arches through the use of one or more tension elements that may be manually and/or automatically adjusted.

Although the present invention has been described in several embodiments, a plenitude of modifications may be suggested to one skilled in the art, and it is intended that the present invention encompass such modifications as fall within the scope of the appended claims.

1. A system for improving a user's breathing, the system comprising:
a mask configured to be positioned on a user's face to deliver gas to a user;
a first oral appliance arch configured to be worn in a user's mouth on a user's mandibular dentition, such that at least a portion of the user's mandibular dentition is received within the first oral appliance arch;
a second oral appliance arch configured to be worn in a user's mouth on a user's maxillary dentition, such that at least a portion of the user's maxillary dentition is received within the second oral appliance arch;
a first tension element coupled to the first oral appliance arch;
a second tension element coupled to the second oral appliance arch;
an adjustment device coupled to the mask and configured to:
receive the first tension element and the second tension element;
adjust, by activating a motor, a position of the first tension element to adjust a forward position of the first oral appliance arch, such that the when the mask is positioned on the user's face and the first oral appliance arch is worn on a user's mandibular dentition, the forward position of the user's mandible is adjusted; and
adjust, by activating a motor, a position of the second tension element independent of the first tension element to position or hold the mask on the user's face; and
a control system configured to control the adjustment device to adjust the position of the first tension element and the second tension element.

2. The system of claim 1, wherein the mask is a multi-chamber mask comprising a nasal chamber and an oral chamber, the nasal chamber separated from the oral chamber by a partition.

3. (canceled)

4. The system of claim 1, wherein the control system comprises a receiver, a processor, and a memory.

5. The system of claim 1, wherein the adjustment device includes a release element configured to release at least the first tension element from the adjustment device.

6. The system of claim 1, wherein the control system is configured to control the adjustment device in response to receiving signals representative of measurement data for one or more of the following:
flow volume for exhaled gases of a user;
pressure of exhaled gases of a user;
breathing rate of a user;
oxygen levels of gases exhaled by a user;
blood oxygen levels of a user;
pulse rate of a user;
vibrations of the mask
vibrations of the adjustment device; and
sound.

7. A system for improving a user's breathing, the system comprising:
a mask configured to be positioned on a user's face to deliver gas to a user;
an adjustment device coupled to the mask and configured to:
receive a first tension element and a second tension element;
adjust, by activating a motor, a position of the first tension element to adjust a forward position of a first oral appliance arch, such that the when the mask is positioned on the user's face and the first oral appliance arch is worn on a user's mandibular dentition, the forward position of the user's mandible is adjusted; and
adjust, by activating a motor, a position of the second tension element independent of the first tension element to position or hold the mask on the user's face.

8. The system of claim 7, wherein the adjustment device is configured to couple to a control system to control the adjustment of the first tension element.

9. The system of claim 7, wherein the mask is a multi-chamber mask comprising a nasal chamber and an oral chamber, the nasal chamber separated from the oral chamber by a partition.

10. The system of claim 7, wherein the second tension element is configured to couple to a second oral appliance arch.

11. The system of claim 7, wherein the adjustment device includes a release element configured to release at least the first tension element from the adjustment device.

12. The system of claim 7, wherein the adjustment device further comprises a manual adjuster configured to adjust the position of a tension element received by the adjustment device in response to manual actuation.

13. An adjustment device for use with a mask and an oral appliance arch, the adjustment device comprising:
a housing with a first opening to receive a first tension element coupled to a first oral appliance arch and a second opening to receive a second tension element coupled to a second oral appliance arch;
an adjustment mechanism within the housing configured to engage the first tension element and the second tension element;
a motor within the housing configured to:
interact with the adjustment mechanism to adjust a position of the first tension element to adjust a forward position of the first oral appliance arch;
interact with the adjustment mechanism to adjust a position of the second tension element independent of the first tension element to position or hold a mask to a user's face.

14. The adjustment device of claim 13, wherein the adjustment mechanism comprises a gear configured to interact with notches in the first tension element, such that rotation of the gear will adjust the position of the first tension element.

15. The adjustment device of claim 13, wherein the adjustment mechanism comprises a threaded element configured to interact with threads in the first tension element, such that rotation of the threaded element gear will adjust the position of the first tension element.

16. The adjustment device of claim 13, wherein the adjustment mechanism comprises a spool configured to receive a portion of the first tension element, such that rotation of the spool will adjust the position of the first tension element.

17. The adjustment device of claim 13, wherein the motor is configured to couple to a control system to control the adjustment of the first tension element.

18. (canceled)

19. The adjustment device of claim 13, further comprising a release element configured to release at least the first tension element.

20. The adjustment device of claim 13, further comprising a manual adjuster configured to adjust the position of a tension element received by the adjustment device in response to manual actuation.

21. A control system for use with a system to improve a user's breathing, the control system comprising:
a receiver configured to receive signals representative of measurement data;
a memory for storing program logic; and
a processor configured to process the received signals and to control an adjustment device in accordance with stored program logic to:
adjust, by activating a motor, a position of a first tension element received within the adjustment mechanism; and
adjust, by activating a motor, a position of a second tension element received within the motorized adjustment mechanism.
